(19) United States
(12) Patent Application Publication
(10) Pub. No.: US 2010/0137266 A1
(43) Pub. Date: Jun. 3, 2010

(54) TREATMENT OF INSULIN RESISTANCE AND DISORDERS ASSOCIATED THEREWITH

(75) Inventors: Eili Tranheim Kase, Oslo (NO); Arild Chr. Rustan, Oslo (NO); Gunn Hege Thoresen, Oslo (NO); Hilde Irene Nebb, Lommedalen (NO); Pål Rongved, Oslo (NO); Jo Klaveness, Oslo (NO); Bjarne Brudeli, Oslo (NO)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITETET I OSLO, BLINDEN (NO)

(21) Appl. No.: 12/444,574

(22) PCT Filed: Oct. 8, 2007
(86) PCT No.: PCT/GB07/03799

§ 371 Date: Feb. 8, 2010

(30) Foreign Application Priority Data
Oct. 6, 2006 (GB) 0619860.0

(51) Int. Cl. A61K 31/575; A61P 3/10; A61P 3/04

(52) U.S. Cl. 514/171; 514/182

(57) ABSTRACT

The present invention provides the use of an LXR antagonist, or a physiologically-acceptable pro-drug therefor, in the manufacture of a medicament for combating insulin resistance or a disorder associated therewith. Further provided is a compound being an ester or carbamate of a hydroxycholesterol, a pharmaceutical composition of such a compound or its use in therapy.

The present invention relates to the use of an antagonist of LXR and in particular a sterol, especially a hydroxycholesterol and most notably 22-S-hydroxycholesterol, or a pro-drug thereof, for the manufacture of a medicament for treating or preventing insulin resistance or disorders associated with therewith, such as for example type 2 diabetes.

Liver X receptors (LXRs) are members of the nuclear receptor superfamily defined as ligand-activated transcription factors. In the presence of their specific ligands nuclear receptors alter the transcription rate of specific genes. LXRα and LXRβ are receptors for oxysterols and are known to play a key role in the regulation of cholesterol metabolism.

LXRα is activated by oxysterols at concentrations which exist in vivo (Janowski et al., Nature, 383, 728-731, 1996). Particularly, LXRα has been shown to be most effectively activated by the oxysterol 24(S), 25-epoxycholesterol which is believed to function as an endogenous activator of LXRα in the liver. However, a range of other oxysterols may also act as agonists of LXRα, for example Forman et al. (PNAS 94, 10588-10593, 1997) report that the oxysterols 20-S-hydroxy-cholesterol (20-S-HC) and 22-R-hydroxycholesterol (22-R—HC) can activate LXRα and relieve the repressive effect of MVA (mevalonic acid) inhibitors on LXRα activity. The synthetic ligand T-0901317 has also been reported as an agonist for LXRα.

As noted above, LXRs are recognised to play a pivotal role in regulating cholesterol efflux, transport or excretion. In particular, LXRs act as a transcriptional master switch for the co-ordinated regulation of genes involved in cellular cholesterol homeostasis, cholesterol transport, catabolism and absorption. A number of genes involved in cholesterol efflux, for example ApoE, ABCA 1 and ABCG1 may be up-regulated by activation of LXRα. This has lead to the proposal to use LXRα agonists as a new strategy for the treatment of cardiovascular disease.

Furthermore, activation of LXRα has been reported to lead to increased glucose uptake and based in part on this LXRα agonists have also been proposed for use in the treatment of diabetes (see for example WO 2004/058175)

In addition to the liver, LXRα may also be expressed in other tissues including skeletal muscle. LXRβ is ubiquitously expressed in adults. The functional role of LXRs in skeletal muscle has up to now been largely unknown. Work leading up to the present invention has now shown that LXRs may play a role in lipid and glucose metabolism in skeletal muscle and other tissues, and more significantly that by antagonising an LXR receptor novel therapeutic benefits may be realised.

Accordingly, we now surprisingly propose a therapeutic utility for antagonists, rather than agonists of LXR, and in particular the use of LXR antagonists for treating or preventing type II diabetes, and other disorders associated with insulin resistance, particularly insulin resistance in skeletal muscle. This is based on the surprising discovery that LXR antagonists may stimulate glucose uptake, notably in skeletal muscle. Thus in patients exhibiting insulin resistance, for example patients suffering from type II diabetes, LXR antagonists may be used therapeutically to increase glucose uptake. Studies with myotubes from type II diabetic patients show reduced glucose uptake in response to LXR activation, as compared to myotubes from healthy subjects. Particularly, hydroxycholesterols are proposed for use according to the present invention as LXR antagonists. Pre-treatment of myotubes with an LXR antagonist (22-(S)-hydroxycholesterol, 22-S-HC, which is shown in our studies to be an LXR antagonist) resulted in a marked stimulation of glucose uptake as compared with the LXR agonists 22-(R)-hydroxy cholesterol (22-R—HC) and T0901317 which show only a slight or no significant effect.

Broadly viewed, the present invention accordingly provides the use of an LXR antagonist, or a physiologically-acceptable pro-drug therefor, in the manufacture of a medicament for combating insulin resistance or a disorder associated therewith.

This aspect of the invention also provides an LXR antagonist, or a physiologically-acceptable pro-drug therefor, for use, in combating insulin resistance or a disorder associated therewith.

In particular, however, the invention provides the use of a hydroxycholesterol, or a physiologically-acceptable pro-drug therefor, in the manufacture of a medicament for combating insulin resistance or a disorder associated therewith.

Thus also provided is a hydroxycholesterol, or a physiologically-acceptable pro-drug therefor, for use in combating insulin resistance or a disorder associated therewith.

As used herein the term “combating” includes both therapeutic treatment and prophylaxis. Thus an LXR antagonist may be used to treat or prevent insulin resistance or a disorder associated therewith. The LXR antagonist may thus be used to treat patients or subjects exhibiting insulin resistance, such as a patient or subject suffering from type II diabetes, or patients or subjects at risk of developing insulin resistance eg. a patient or subject at risk of developing type II diabetes.

“Insulin resistance” is defined as the impaired ability of insulin (either endogenous or exogenous) to reduce blood glucose. Particularly, the present invention is concerned with insulin resistance in skeletal muscle. In skeletal muscle insulin resistance may be characterised by impaired insulin-mediated reduction in glucose uptake, impaired insulin-mediated glycogen synthesis and glucose oxidation, lower lipid oxidation and increased intracellular lipid content. There may also be mitochondrial dysfunction and down-regulation of genes involved in oxidative phosphorylation. In particular, according to the present invention, by stimulating glucose uptake, the LXR antagonist acts to counteract the effect of insulin resistance in reducing glucose uptake.

Hence, disorders associated with insulin resistance may result from the reduced ability of insulin to lower blood glucose. The term “disorder associated with insulin resistance” as used herein includes any disorder or condition in which insulin resistance is exhibited or manifest, as may be determined for example by a reduced ability to take up glucose eg. from the blood or by tissues such as skeletal muscle, or any disorder or condition which may lead to or cause insulin resistance. Insulin resistance may be determined by raised plasma insulin concentrations, in the presence of normal or increased glucose concentrations.

Insulin resistance is characterized by a raised insulin plasma concentration, in the presence of normal or increased glucose levels. A surrogate measure for insulin resistance, which can be used in patient cohorts, is the Homeostasis Model Assesment Method for insulin resistance (HOMA-IR, Wallace 2004). For clinical studies, a hyperinsulinemic euglycemic clamp is the golden standard to measure insulin resistance. In addition, an improved glucose uptake in combination with a reduced insulin resistance is also likely to improve glucose tolerance, which can be measured with an oral glucose tolerance test, using the WHO 1999 criteria (Definition, diagnosis and classification of diabetes mellitus and its complications). For example: for early detection of DM2 or the prediabetic state, WHO recommends to perform a standard oral glucose tolerance test. In this test, blood glucose is measured during fasting and 2 hours after the intake of a 75 g-glucose load, dissolved in 250 ml of water. The test is preferably performed twice. Subjects can be classified as having Impaired Glucose Tolerance if fasting venous plasma concentrations exceeds 7.0 mmol/l or are in the range between 7.8 and 11.1 mmol/l 2 hours after glucose intake.

Disorders associated with insulin resistance include Type II diabetes and the treatment or prevention of type II diabetes represents a preferred aspect of the present invention

Type II diabetes is well documented in the art and is also known as adult-onset or noninsulin-dependent diabetes mellitus (NIDDM). Type II diabetes is a chronic disease that occurs when the body resists insulin and plasma glucose levels remain high. The body thus has an inability to deal with increased plasma glucose levels. Insulin resistance in skeletal muscle is a salient feature of type II diabetes and visceral and ectopic fat depots are often increased.

A further preferred aspect of the invention is the treatment of obesity or subjects at risk of obesity. Obese individuals may often exhibit insulin resistance, particularly insulin resistance in skeletal muscle.

The term “obesity” as used herein refers to individuals who are overweight or obese. Such individuals may have a body mass index of greater than 25, where the body mass index is calculated by comparing weight to height by dividing the weight measurement (expressed in kilograms) by the square of the height (expressed in metres). Overweight individuals may have a body mass index of 25 to 30, obese individuals may have a body mass index of over 30, and morbidly obese individuals may have a body mass index of over 35. Obese individuals as referred to herein may have varying fat distribution and in particular, the present invention relates to the treatment of individuals with central or truncal obesity, where excess fat is located in the abdomen. This may be determined for example by measuring waist circumference. Waist circumference thresholds indicating central obesity may be taken as greater than 102 cm, or more particularly as greater than 94 cm for men and greater than 88 cm or more particularly greater than 80 cm for women for people of white Northern European extraction. For people from other ethnic groups e.g. South Asian and Chinese, these values may be reduced e.g. >90 cm for men and >80 cm for women. The measurement of body fat distribution and content is also discussed in Goodpaster 2002 Curr. Opin. Clin. Nutr. Metab. Care 5: 481-487, and include for example in vivo imaging modalities such as computerised tomography and MRI, for example to quantify region-specific fat distribution. Magnetic resonance spectroscopy may be used to, directly quantify the content or concentration of lipid in tissue. Finally, direct quantification of lipid contained in tissue may be performed from extracted tissue samples, through a biopsy.

As noted above, disorders associated with insulin resistance include disorders or conditions which cause insulin resistance. Insulin resistance is linked with lipid accumulation and may result from obesity. Thus obesity may be manifest, but the individual may not yet exhibit insulin resistance. An obese individual may be at risk of developing insulin resistance. More particularly, ectopic lipid accumulation, that is lipid accumulation in organs or tissues, eg, in muscle, liver and pancreas may cause insulin resistance. Thus, a person exhibiting or suffering from tissue lipid accumulation may be “fat” on the inside without being classified as “fat” on the outside. Such a person may not yet be classified as obese, or even overweight, but nonetheless may be at risk of developing insulin resistance, or type II diabetes. Such a tissue (or ectopic) lipid accumulation is recognised as being highly unhealthy. In addition to the effects reported above, the present invention further provides a rationale for treating such patients using LXR antagonists. Thus, in addition to the effect in stimulating glucose uptake, LXR antagonists may also have effects on lipid metabolism in skeletal muscle. In particular, LXR antagonists may reduce the synthesis of lipids, leading to decreased lipid accumulation. More particularly, LXR antagonists may reduce the synthesis of triacyl- and diacyl-glycerols (TAGs and DAGs). As will be described in more detail below, the LXR antagonist 22-S-HC has been shown to reduce TAG synthesis from palmitate and DAG synthesis from acetate. This is believed to result from the repression of certain genes involved in cellular/tissue lipid accumulation. As reported below, 22-S-HC represses or down-regulates the expression of CD36, SCD-1, ACSL1 and FAS. In particular it is believed that LXRα antagonists may reduce lipid formation, especially TAG and DAG formation, by repressing the expression (eg.mRNA levels) of the above genes, particularly SCD-1 and ACSL1.

LXR antagonists may thus be used to reduce lipid formation and/or lipid accumulation in ectopic tissues, for example skeletal muscle. They may thus be used to combat tissue lipid accumulation, both in obese and non-obese individuals, and accordingly the combatting of tissue lipid accumulation represents a further preferred aspect of the present invention.

Thus, in a further aspect the present invention provides the use of an LXR antagonist, especially a hydroxycholesterol, or a physiologically-acceptable pro-drug therefor, in the manufacture of a medicament for combatting tissue lipid accumulation.

This aspect of the invention also provides an LXR antagonist, especially a hydroxycholesterol, or a physiologically-acceptable pro-drug therefor, for use in combatting tissue lipid accumulation.

Tissue lipid accumulation may be assessed or determined by tests known in the art and described in the literature, for example in the Goodpaster 2002 review (supra). Such methods may include, as mentioned above, in vivo imaging eg. by CT or MRI, whole body magnetic resonance spectroscopy and direct quantification of lipids in biopsy samples.

Tissue lipid accumulation may occur at different sites in the body, most notably the skeletal muscles, but also the liver and pancreas. Biopsy samples of myotubes from skeletal muscle may be assessed as above for lipid content or composition. The tissue lipid accumulation may result from an increased synthesis of DAG and/or TAG. Hence, tissue lipid accumulation may be determined by measuring the levels of DAG and/or TAG or other accumulated lipids at a particular site.

The term “LXR antagonist” includes any agent, which may include any compound, substance or molecule, capable of antagonising any function of an LXR receptor. An antagonist may thus antagonise (down-regulate, inhibit or suppress) any effect of LXR activation.

An LXR antagonist according to the present invention, may be an antagonist of LXRα or LXRβ or both. Thus an LXRα antagonist may be used.

As noted above, hydroxycholesterols are proposed according to the present invention as LXR antagonists, particularly LXRα antagonists.

More particularly, according to the present invention an LXR antagonist may have the effect of stimulating or increasing glucose uptake, for example in human or other animal myotubes, as compared to basal glucose uptake or compared to an LXR agonist such as 22-R-HC or T0901317. Briefly, myotube cultures may be pre-treated with the antagonist or test or control compound (eg. for a period of days, eg. 4 days) The cultures may then be exposed to labelled glucose (eg. for 4 hours) to study glucose uptake. A procedure for such a test is described in the Examples below. In particular, an antagonist according to the present invention may exhibit at least 50%, more particularly at least 60, 65, 70 or 75% of the activity of 22-S-HC in stimulating glucose uptake by myotubes (eg. healthy human myotubes).

An LXR antagonist may alternatively be identified or assessed by means of its effect on lipid metabolism. Thus, according to the present invention an antagonist may reduce lipid formation, for example in myotubes (eg. healthy human myotubes) as compared to basal lipid formation, or as compared to an LXR agonist such as 22-R—HC or T0901317. Myotube cultures may be pre-treated with antagonist or test

or control compounds as above, and then exposed (eg. for 4 hours) to labelled substrate for lipid formation eg. acetate or palmitate. Lipids may be separated from the culture eg by TLC, and identified. More specifically, the formation of specific lipids may be assessed, for example TAG and/or DAG eg. the formation of TAG from palmitate and/or the formation of DAG from acetate. A procedure for such a test is described in the Examples below. In particular, an antagonist according to the present invention may exhibit at least 50%, more particularly at least 60, 65, 70 or 75% of the activity of 22-S-HC in reducing lipid formation by myotubes (eg. healthy human myotubes) eg. lipid eg.TAG formation from palmitate and/or lipid eg.DAG formation from acetate.

An LXR antagonist may be identified or assessed by means of its effect in repressing the expression of target LXR genes, particularly genes involved in lipid or fatty acid metabolism. An antagonist may accordingly repress the expression of the genes fatty acid transporter CD36 (CD36), stearoyl-CoA desaturase-1 (SCD-1), acyl CoA synthetase long chain family member-1 (ACSL1) and/or fatty acid synthase (FAS), particularly SCD-1 and/or ACSL1. Methods for assessing gene repression are well known in the art and include for example reverse transcription of total mRNA and real-time quantitative PCR using specific primers. A procedure for this is described in the Example below. In particular, an antagonist according to the present invention may exhibit at least 50%, more particularly at least 60, 65, 70 or 75% of the activity of 22-S-HC in reducing expression of CD36, SCD-1, ACSL1 and/or FAS by myotubes (eg. healthy human myotubes).

An LXR antagonist may also be identified by virtue of its ability to repress the effects or expression of other LXR target genes. Furthermore, antagonist activity may be detected or identified by coupling the expression of a reporter gene to a promoter or response element of an LXR target gene, for example luciferase expression may be assessed coupled to a FAS promoter fragment, as described in Example 1 below, and determining whether expression of the reporter gene is reduced.

Antagonists may also be identified by determining whether they can reduce or abolish the effects of a known LXR agonist such as T0901317.

Other tests for LXR antogonists may be also be used, according to procedures or principles known in the art or described in the literature. Thus for example a ligand-sensing assay, which measures ligand-dependant recruitment of a peptide from the steroid receptor coactivator 1 (SRC1) to the LXRα receptor, may be used as follows:

A modified polyhistidine tag (MKKGHHHHHHG) is fused in frame of the human LXRα ligand-binding domain (amino acids 183-447 of GenBank accession number U22662, with the 14th amino acid corrected to A from R). The LXRα fusion protein is expressed in E. coli and purified as described in Parks, D. J. et al, Science 1999, 254, 1365-1368 and Janowski, B. A. et al., Proc. Natl. Acad. Sci. USA, 1999, 95, 256-271. The purified protein is diluted to approximately 10 μm in PBS and a 5-fold molar excess of NHS-LC-Biotin (Pierce) is added in a minimal volume of PBS. This solution is incubated with gentle mixing for 30 min at ambient room temperature. The biotinylation reaction is stopped by the addition of 2000-fold molar excess of Tris-HCl, pH8. The modified LSRα protein is dialyzed against 4 buffer changes, each of at least 50 volumes, with PBS containing 5 mM DTT, 2 mM EDTA and 2% sucrose. The biotinylated LXRα protein is subjected to mass spectrometric analysis to reveal the extent of modification by the biotinylation reagent. In general, approximately 95% of the protein has at least a single site of biotinylation; the overall extent of biotinylation followed a normal distribution of multiple sites, ranging from 1 to 9.

The biotinylated protein is incubated for 20-25 min at a concentration of 20 nM in assay buffer (50 mM NaF, 50 nM MOPS, pH 7.5, 0.1 mM CHAPS, 0.1 mg/mL FAF-BSA, 10 mM DTT) with equimolar amounts of streptavidin-AlloPhyeoCyanin (APC, Molecular Probes). At the same time, a biotinylated peptide comprising amino acids 675-699 of SRC1 9CPSSHSSLTERHKILHRLLQEGSPS-CONH₂) at a concentration of 20 nM is incubated in assay buffer with an equimolar amount of streptavidin-labeled europium (Wallac) for 20-25 min. After the initial incubation is completed, a 20 molar excess (400 nm) of biotin is added to each of the solutions to block the unattached streptavidin reagents. After 20 min at room temperature, the solutions are mixed, yielding a concentration of 10 nM for the dye-labeled LXRα protein and SRC1 peptide. 49 μL of the protein/peptide mixture is added to each well of an assay plate containing 1 μL of test compound. The final volume in each well was 0.05 mL, and the concentration in the well for the dye-labeled protein and peptide is 10 nM. The final test compound concentrations may be between 1 nM and 100 μM. The plates are incubated at room temperature for 2-4 h and then counted on a Wallac Victor fluorescent plate reader in a time-resolved mode. The relative fluorescence is measured at 665 nm.

LXR antagonists for use according to the present invention can be known agents that antagonise the LXR receptor or derivatives thereof or novel antagonists can be identified by screening for antagonist activity as indicated above. LXR antagonists can therefore be small organic molecules, peptides or polypeptides, nucleic acids or other agents. They may be naturally occurring molecules or synthetic molecules. Test agents for screening may be obtained from a variety of sources eg. compound libraries, for example combinatorial libraries or peptide libraries such as phage display libraries, which may be generated according to procedures or principles well known in the art, or from libraries of natural compounds eg. in the form of bacterial, plant, fungal and animal extracts which can be obtained from commercial sources or collected in the field.

The antagonists may also be obtained by rational design, for example based on known antagonist structures. Known antagonists or other agents may be subjected to directed or random chemical modification to produce structural analogues.

An LXR antagonist according to the invention may be a sterol, particularly an oxysterol. More particularly the antagonist may be a sterol (e.g. oxysterol), with oxidation of the sterol side chain. Preferred sterols are a cholesterol or a cholenamide, particularly a cholesterol. The sterol preferably carries a functional hydrogen bond acceptor on the sterol chain, preferably a hydroxy group. Thus the antagonist may be a hydroxycholesterol or hydroxycholenamide, and is preferably a hydroxycholesterol. Especially preferred is a hydroxycholesterol carrying one or more hydroxy groups on any one or more of the carbon atoms in the sterol side chain eg. from C20 to the end of the sterol side chain, for example at any one or more of C20 to C27.

Preferably, the hydroxy group is at position 20, 22, 23, 24, 25, 26 or 27, more preferably at position 20, 22, 24 or 25. Preferred are hydroxycholesterols with a hydroxy group at or adjacent to position 22.

The antagonistic effect may be dependent on, or specific to, a particular stereochemistry. Thus, for a given hydroxy-group position, the hydroxycholesterol may be the R or the S enantiomer. It is preferred that for a given position, the stereochemistry is the opposite of the stereochemistry of the endogenous hydroxycholesterol, i.e. the endogenous equivalent. Thus, for example, where the endogenous hydroxycholesterol is 24-S-HC or 20-S-HC, the LXR antagonist may be 24-R-HC or 20-R-HC. Similarly, where a particular hydroxycholesterol is known or shown to activate LXR, (e.g. 22-R-HC, 24-S-HC, 20-S-HC, 20-R, 22-R-diHC, 24(S), 25 epoxycholesterol, 23-S-HC, etc.) the opposite enantiomer may be an LXR antagonist (e.g. 22-S-HC, 24-R-HC, 20-R-HC, etc).

The cholesterol moiety of the hydroxycholesterol may be modified, for example by ring substitution or unsaturation of the B ring. The length of the sterol side chain may be modified.

Hydroxycholesterols and their various enantiomers and methods for their synthesis are well known and widely described in the art.

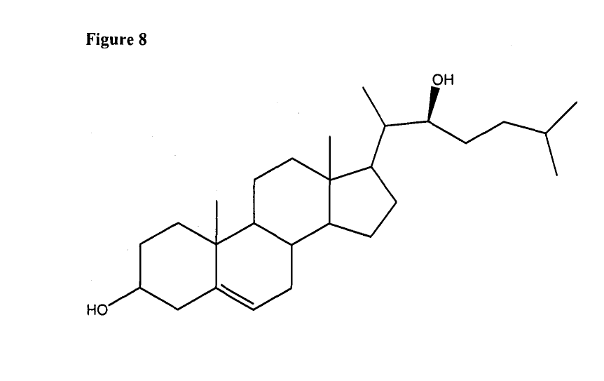

Especially preferred as an antagonist according to the present invention is 22-S-hydroxycholesterol as shown in FIG. 8. 22-S-HC is available commercially (e.g. from Sigma) and its synthesis has been described in the art (see Burrows et al. J. Org. Chem. 1969, 34(1),103-107). Also encompassed are derivatives of 22-S-hydroxycholesterol, e.g. with a modified cholesterol moiety as noted above or a modified side chain. Such derivatives retain the activity of 22-S-HC i.e. LXR antagonist activity.

The hydroxycholesterol, e.g. 22-S-HC or any other LXR antagonist, may repress or downregulate any one or more of the genes CD36, SCD-1, ACSL1 and FAS, particularly SCD-1 and/or ACSL1. Preferably, the hydroxycholesterol (e.g. (22-S-HC) or any other LXR antagonist can repress or downregulate these genes by 30, 40, 50, 60, 70, 80 or 90% compared to expression of these genes in the absence of 22-S-HC or other antagonist. Such downregulation or repression can be determined by measuring mRNA levels produced from the genes .e.g. by Northern blotting or Real Time PCR. More particularly, the hydroxycholesterol (e.g. 22-S-HC) or any other LXRα antagonist can repress or downregulate anyone of and preferably both of SCD-1 or ACSL1 by 30, 40, 50, 60, 70, 80 or 90% compared to the expression of the genes in the absence of 22-S-HC or any other LXR antagonist.

Further, preferably the hydroxycholesterol (e.g. 22-S-HC) or any other LXR antagonist may reduce the synthesis of DAG and/or TAG by 30, 40, 50, 60, 70, 80 or 90% compared to synthesis in control untreated cells. DAG and/or TAG synthesis can be measured by investigating the incorporation of labelled acetate and/or palmitate into DAG and/or TAG in the presence or absence of the hydroxycholesterol (e.g. 22-S-HC) or any other LXR antagonist.

Other antagonists which may be used include naturally occurring LXR antagonists such as polyunsaturated fatty acids particularly n-3 fatty acids and geranyl geraniol or geranylgeranyl pyrophosphate Other antagonists include 5α,6α-epoxycholesterol sulphate (ECHS) and 7-ketocholesterol-3-sulphate (Song et al. Steroids 2001 66(6) 473-9). Thus sulphated oxysterols may be used as antagonists.

As indicated above the LXR antagonist may be supplied in the form of a pro-drug, or bioprecursor. Such a pro-drug may have protected functional groups eg. protected hydroxy groups. The protecting group is metabolically cleavable to release the active or parent compound in the body. Preferably the pro-drug is water soluble or has improved water solubility relative to the parent compound.

The prodrug may be transformed in vivo to the active compound (eg. hydroxycholesterol) by an enzymatic transformation or hydrolytic reaction. Thus the prodrug may be transformed by esterases, amidases and/or oxidative enzymes.

A prodrug according to the invention may thus comprise at least one of the following functional groups: esters, including carbonate esters, carbamates, ethers and acetals and alkoxy groups.

Preferred prodrugs are in the form of esters or carbamates.

The preferred hydroxysterol antagonists of the invention may accordingly be in the form of an ester (eg. a double ester) at the hydroxy group eg at the 3-hydroxy group and/or a hydroxy group on the 17-alkyl group (the sterol side chain).

Esters may be formed preferably with di-acids (eg. short chain di-acids) or hydroxy-acids or acids with other solubilizing groups (eg (poly)hydroxy, (poly)ether, amine, thiol, etc), for example amino acids. In particular, an acid is used which is physiologically tolerable, e.g. azelaic acid, glutaric acid, succinic acid, or glycine or derivatives thereof (e.g. N-(tert-butoxycarbonyl)glycine) so that ester cleavage of the pro-drug releases the parent drug and a physiologically tolerable acid metabolite.

Alternatively, amino acids may be used to form carbamates at hydroxy groups. Thus in the case of hydroxycholesterols, carbamates may be formed with amino acids at the 3-hydroxy group and/or at a hydroxy group on the sterol side chain.

As indicated above, amino acids may be used, inter alia, also to form esters at hydroxy groups. Amino acid-based pro-drugs may have the advantage of increased tissue-uptake as compared with the parent drug. As noted above, such amino acids may include glycine or derivatives thereof, e.g. N-(tert-butoxycarbonyl) glycine.

As noted above, the preferred hydroxycholesterol antagonists of the invention may also be in the form of ether, acetal or alkoxy derivatives

Water solubility may further be enhanced using water-soluble counter-ions to the carboxylic acid functionality.

Methods for preparing such esters and carbamates and appropriate acids to use are well known in the art. The esters or carbamates may be formed optionally during production of the hydroxysterol or other antagonist, or optionally afterwards. If ester or carbamates formation at only one or selected hydroxy groups is required, then selected hydroxy groups may optionally be protected before esterification or carbamate formation and deprotected afterwards.

Similarly, ether, acetal or alkoxy prodrugs may be prepared according to methods known in the art using similar principles eg either during production of the hydroxycholesterol or other antagonist, or optionally afterwards. If ether, acetal or alkoxy formation at only one or selected hydroxy groups is required, then selected hydroxy groups may optionally be protected during the reaction and deprotected afterwards.

A prodrug of a hydroxycholesterol may be a compound of formula I or a physiologically acceptable salt thereof:

L)ₙ-O—CH₂—OW Ia

L)ₙ-O—W Ib

L)ₙ-O—CO(NH)ₐ—(O)_{b}—W Ic

wherein,
L-OH is a hydroxycholesterol,
n is a positive integer or a positive fraction,
a=0 or 1, b=0 or 1 and a+b=0 or 1, and
W is a linear, branched or cyclic, saturated or unsaturated organic group that comprises up to 25 carbon atoms and optionally incorporates heteroatoms (e.g. O, N and/or S).

W may thus be an organic group with a carbon backbone and may for example be an aliphatic, alicyclic or aromatic group eg. a linear alkylene chain. W may for example be any, optionally substituted, alkyl, aryl, alkenyl or alkynyl group.

More particularly W may be an alkyl (e.g. C₁₋₆ alkyl) or a C₁₋₆ alkylene chain substituted by an COOH or NH₂ group.

A representative prodrug formula for a hydroxysterol according to the present invention may thus be:

L)ₙ-O—CO(NH)ₚ—R—XY (Formula II)

wherein

L-OH=sterol;

n=positive integer, eg. 1 or 2;

p=0 or 1;

HO—COR(X)ₘ Y=an acid or salt, amide or ester thereof, preferably an acid or di-acid or salt thereof; or

HO—CONHR(X)ₘY=an acid or salt, amide or ester thereof, preferably an acid or di-acid or salt thereof;

X=a solubilizing group e.g. an acid (e.g. carboxyl), hydroxy, amino or thiol, or polyether group;

m=zero or positive integer, e.g. 1-6, especially 1;

R is an acid skeleton, which may be linear, branched or cyclic, saturated or unsaturated (e.g. aromatic), and may comprise up to 25 carbons, especially up to 10 or 8 carbons (preferably a linear alkylene chain), optionally incorporating heteroatoms, for example selected from O, N and S;

Y=hydrogen(s) or a physiologically tolerable counterion(s), eg Na⁺, K⁺, Ca²⁺, Mg²⁺, meglumine, halide, etc, or a hydrophobic amino alcohol possessing a cation on the amino group at physiological pH, eg. tris or meglumine.

Group R as noted above is an acid skeleton. This may be defined as a group which serves as the framework to carry the acid groups which carry the sterol and any such other solubilising groups as are desired. R may thus be a linear, branched, cyclic, saturated or unsaturated organic group which may comprise up to 25 carbon atoms (or up to 10 or 8 carbon atoms), optionally incorporating heteroatoms (eg. O, N or S). R is thus an organic group with a carbon backbone and may for example be an aliphatic, alicyclic or aromatic group eg. a linear alkylene chain. By way of representative example, R may be a group such that the sterol is coupled to succinic acid, or glutaric acid or glycine or a derivative thereof.

More particularly, in the case of an ester with a di-acid, X may be a carboxylic acid moiety which is in anionic form at physiological pH; in the case of a hydroxy acid ester, R—X Y may be a polyhydroxyalkyl group, eg. with 3-6 hydroxy groups; and in the case of an amino acid ester, X may be an amino functionality —NHR₁, wherein R₁ may be a H or a lower (eg. 1-6 or 1-4) alkyl eg. methyl, X being cationic at physiological pH.

Where X is NHR₁, the amine may be present as its free form or as salt where Y is methane sulphonic acid or any other suitable counterion.

The prodrug as described above takes the form of one or more sterol moieties coupled cleavably to one or more acid moieties. Thus, the prodrug may of course have more than one cleavable acid attached to each sterol moiety. In Formula I above n may thus be a positive fraction (e.g. ½, ⅓ etc) or a positive integer.

In a preferred embodiment the prodrug may accordingly be a compound of formula III or a physiologically acceptable salt thereof:

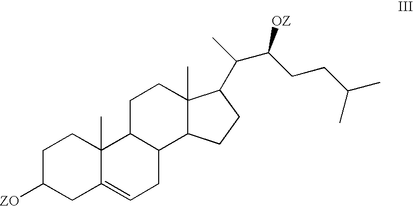

wherein,
each Z may be the same or different and is selected from H or CO(NH)ₐ—(O)_{b}—W wherein a=0 or 1, b=0 or 1 and a+b=0 or 1 and W is a linear, branched or cyclic, saturated or unsaturated organic group that comprises up to 25 carbon atoms and optionally incorporates heteroatoms (e.g. O, N and/or S).

As noted above, W may preferably be alkyl (e.g. C₁₋₆ alkyl) or a C₁₋₆ alkylene chain substituted by an COOH or NH₂ group.

A representative hydroxycholesterol pro-drug may be the monoester mono-acid derivative of succinic acid and 22-S-HC, or a salt thereof, the diester derivative of succinic acid and 22-S-HC or a salt thereof, the mono- or diester derivative of glutamic acid and 22-S-HC, or the mono- or diester derivative of 22-S-HC with glycine or a derivative thereof (e.g. (N-tert-butoxycarbonyl)glycine.

Pro-drugs of antagonists according to the present invention, particularly esters and carbamates of antagonists such as sterols, particularly hydroxycholesterols, and especially 22-S0-HC, represent novel chemical entities. Thus, in a further aspect the present invention provides a compound being an ester or carbamate of a hydroxycholesterol, and particularly such an ester or carbamate for use in therapy.

Also provided is a pharmaceutical composition comprising a prodrug of an LXR antagonist together with at least one pharmaceutically acceptable diluent or carrier.

More particularly, the composition comprises an ester or carbamate or ether or acetal or alkoxy derivative of a hydroxycholesterol.

Preferably the pro-drug is a compound of Formula I and most preferably a carbamate or ester or ether or acetal or alkoxy derivative of 22-S-HC.

Compositions comprising the LXR antagonist or pro-drug thereof are preferably formulated prior to administration. The active ingredients in such compositions may comprise from 0.05% to 99% by weight of the formulation.

Appropriate dosages may depend on the antagonist to be used, precise condition to be treated, age and weight of the patient etc. and may be routinely determined by the skilled practitioner according to principles well known in the art. By way of example, representative dosages may include 1 to 200 or 1-100 mg/kg eg. 5 to 70, 5-50, or 10 to 70 or 10 to 50 mg/kg.

By “pharmaceutically acceptable” is meant that the ingredients must be compatible with other ingredients of the composition as well as physiologically acceptable to the recipient.

Pharmaceutical compositions for use in methods according to the present invention may be formulated according to techniques and procedures well known in the art and widely described in the literature and may comprise any of the known carriers, diluents or excipients. Other ingredients may of course also be included, according to techniques well known in the art e.g. stabilisers, preservatives, etc. The formulations may be in the form of sterile aqueous solutions and/or suspensions of the pharmaceutically active ingredients, aerosols, ointments and the like. The formulations may also be in a sustained release form e.g. microparticles, nanoparticles, emulsions, nanosuspensions, lipid particles or oils. Further, films, patches or folios having the LXR antagonist coated on the surface may also be used in the present invention.

The administration may be by any suitable method known in the medicinal arts, including oral, parenteral, topical, subcutaneous administration or by inhalation. The LXR antagonist or prodrug or formulations comprising the LXR antagonist or prodrug thereof may be administered in a single dose to be taken at regular intervals e.g. once or twice a day, once every 48 hours or once every 72 hours. Sustained formulations may be given at longer intervals e.g. 1 to 2 times a month or every three months

The precise dosage of the active compounds to be administered, the number of daily or monthly doses and the length of the course of treatment will depend on a number of factors, including the age of the patient and their weight.

The compositions may be formulated according to techniques and procedures well known in the literature and may comprise any of the known carriers, diluents or excipients. For example the compositions/formulations which can be used in the present invention which are suitable for parenteral administration conveniently may comprise sterile aqueous solutions and/or suspensions of pharmaceutically active ingredients preferably made isotonic with the blood of the recipient generally using sodium chloride, glycerin, glucose, mannitol, sorbitol and the like. In addition, the composition may contain any of a number of adjuvants, such as buffers, preservatives, dispersing agents, agents that promote rapid onset of action or prolonged duration of action.

Compositions/formulations suitable for oral administration may be in sterile purified stock powder form, preferably covered by an envelope or envelopes which may contain any of a number or adjuvants such as buffers, preservative agents, agents that promote prolonged or rapid release.

Compositions/formulations for use in the present invention suitable for local or topical administration may comprise the LXR antagonist or prodrug mixed with known suitable ingredients such as paraffin, vaseline, cetanol, glycerol and its like, to form suitable ointments or creams.

In addition to formulation as pharmaceutical compositions, the LXR antagonists may be provided according to the present invention as or in functional foods. Thus, the LXR antagonist may be added to or included in foodstuffs or food products eg. milk, yoghurt or other dairy products, breakfast cereals or bakery products or in drinks, beverages etc.

In a further aspect the invention provides a method of combating insulin resistance or a disorder associated therewith comprising the step of administering an LXR antagonist, especially a hydroxycholesterol, or a pro-drug therefor to an individual in need thereof.

Such an individual may be a human or a non-human animal subject.

A further aspect of the present invention also provides a pharmaceutical composition comprising an LXR antagonist, especially a hydroxycholesterol, or a physiologically acceptable pro-drug therefor, together with at least one pharmaceutically acceptable diluent or carrier for use in combating insulin resistance or a disorder associated therewith.

Another preferred aspect of the present invention relates to combination of an LXR antagaonist or prodrug therefor, particularly a hydroxycholesterol or prodrug therefor, preferably 22(S)-hydroxycholesterol or a 22(S)-hydroxycholesterol prodrug, with other therapeutic agents which are directly or indirectly related to insulin resistance or disorders associated with insulin resistance.

A further aspect of the present invention thus provides a product comprising an LXR antagonist, particularly a hydroxycholesterol (eg. 22-S-HC), or a physiologically acceptable prodrug therefor, and a second agent effective for combating insulin resistance or a disorder associated therewith as a combined preparation for simultaneous, separate or sequential use in combating insulin resistance or a disorder associated therewith.

As noted above such a second agent may directly or indirectly be useful in combating insulin resistance or a disorder associated therewith eg. in treating said insulin resistance or disorder associated therewith.

The antagonist (eg. hydroxycholesterol, eg. 22-S-HC) or prodrug therefore may be administered together in the same composition or separately in separate compositions. They may be administered at the same time or separately eg sequentially, for example at spaced intervals.

A further aspect of the invention thus also provides a pharmaceutical composition comprising an LXR antagonist, particularly a hydroxycholesterol (eg. 22-S-HC), or a physiologically acceptable prodrug therefor, and a second agent effective for combating insulin resistance or a disorder associated therewith, together with at least one pharmaceutically acceptable diluent or carrier.

An exemplary second agent includes a HMG CoA reductase inhibitor or other specific inhibitor of cholesterol synthesis. Such HMG CoA reductase inhibitors include for example the so-called statins, for example simvastatin, atorvastatin, lovastatin and fluvastatin. Also, other drugs that inhibit cholesterol synthesis at a later stage in the metabolic pathway may be used, for example sqalene synthesis inhibitors.

Other exemplary second agents include drugs with an effect on the peroxisome proliferators-activated receptor (PPAR). Typical such drugs include rosiglitazone, pioglitazone, clofibrate, rivoglitazone and fenofibrate.

Other second agents include agents for the treatment of type-2 diabetes. These include insulin, metformin alpha-glucosidase inhibitors (e.g. Akarbose, voglibose), glinides (e.g. repaglinid, nateglinid) and sulfonurea drugs, for example tolbutamide, glimepiride, glibenclamide, chlorpropamide and glyhexamide. These also include drugs with an effect on the incretin-system, for example incretin mimetics (e.g. exenatide, liraglutide, exenatide LAR) and dipeptidylpeptidase inhibitors (e.g. sitagliptin, vildagliptin, saxagliptin, alogliptin).

Further exemplary second agents include drugs for treatment of obesity. These may include pancreatic lipase inhibitors eg. orlistat, or CNS-related appetite-reducing substances eg. sibutramine, drugs with an effect on the angiotensin-renin system eg. ACE inhibitors, for example captopril and enalapril, angiotensin II receptor antagonists eg. losartan, valsartan, candesartan and irbesartan. Further exemplary second agents include, anti-inflammatory agents eg. glucocorticoids and non-steroid anti-inflammatory agents (NSAIDs), cholinesterase inhibitors and N-methyl D-aspartate receptor (NMDA) antagonists.

The invention will now be described in more detail in the following non-limiting Examples, with reference to the following drawings:

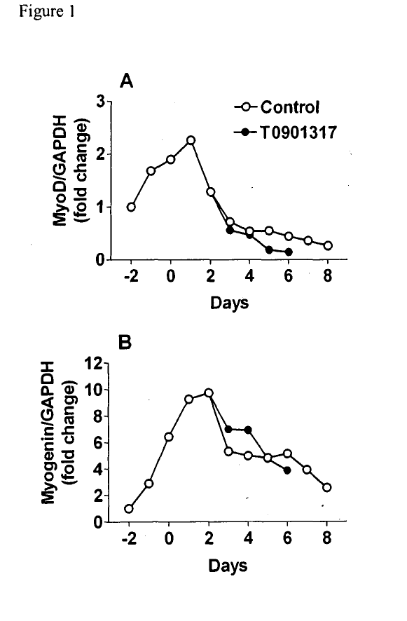

FIG. 1. Expression of MyoD (A) and myogenin (B) during differentiation of myoblasts. During the differentiation process, cells were harvested on day—2 until day 8. Some cell cultures were treated with ±1 μM T0901317 from day 2 until day 6. Equal amount of total RNA from each donor (n=4) were pooled, reversely transcribed and analyzed by Real-Time RT-PCR. Expression of MyoD and myogenin was normalized to GAPDH.

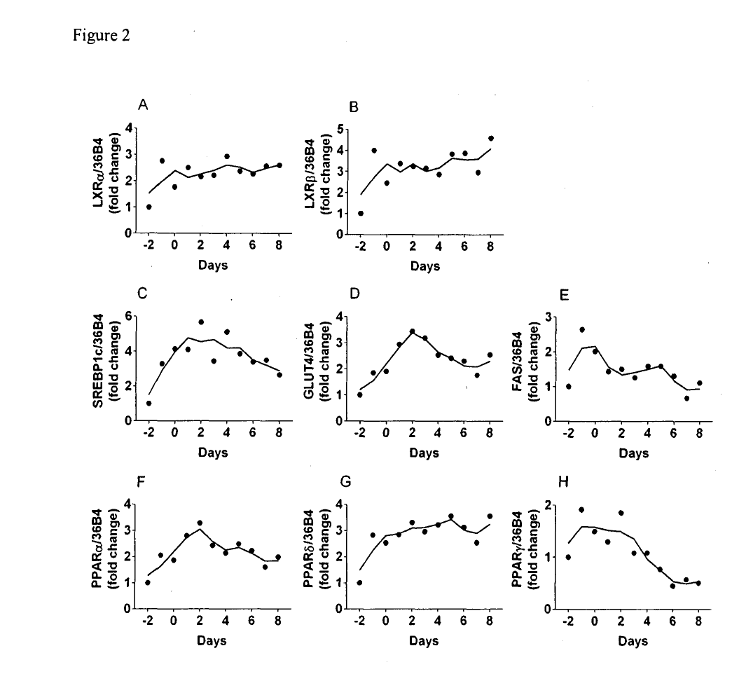

FIG. 2. Expression of LXRs and known target genes during myotube differentiation. During the differentiation process, cells were harvested on day—2 until day 8. Equal amount of total RNA from each donor (n=4) were pooled, reversely transcribed and analyzed by Real-Time RT-PCR. The mRNA expressions were normalized to 36B4. Relative expression of (A) liver X receptor (LXR)α, (B) LXRβ, (C) sterol regulatory element-binding protein (SREBP)1c, (D) GLUT4, (E) fatty acid synthase (FAS), (F) peroxisome proliferator-activated receptor (PPAR)α, (G) (PPAR)δ, and (H)PPARγ.

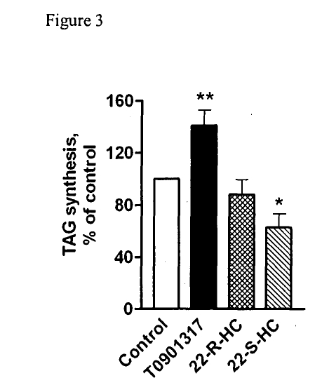

FIG. 3. 22-hydroxycholesterols influence TAG synthesis from palmitic acid differently than T0901317. Human myoblasts were allowed to differentiate for 2 days, and then exposed to vehicle (0.1% DMSO), 1 μM T0901317, or 10 μM 22-S-hydroxycholesterol (22-S-HC) for 4 days. Differentiated myotubes were then incubated with [1-¹⁴C]PA (0.5 μCi/ml, 0.1 mM) for 4 h before triacylglycerol (TAG) levels were determined. Results present means±SEM (n=4; independent muscle cell donors). *p<0.05 vs control, **p<0.05 vs all other treatments.

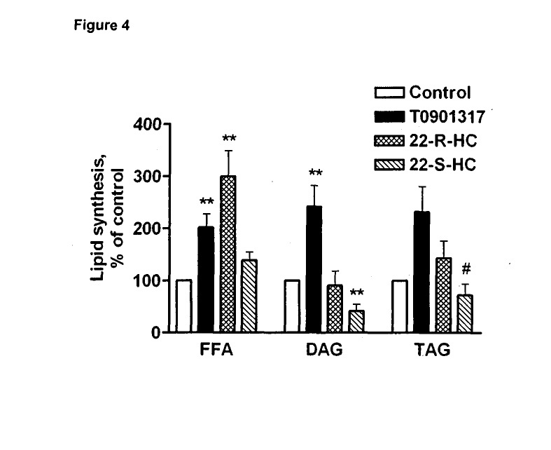

FIG. 4. 22-hydroxycholesterols influence lipid formation from acetate differently than T0901317. Human myoblasts were differentiated and treated with LXR ligands as described in FIG. 3. The cells were then incubated with [1-¹⁴C]acetate (2 μCi/ml, 0.1 mM) for 4 h before levels of free fatty acids (FFA), diacylglycerol (DAG) and triacylgycerol (TAG) were determined. Results present means±SEM (n=5; independent muscle cell donors). **p<0.05 vs all other treatments, ^{#}p<0.05 vs T0901317.

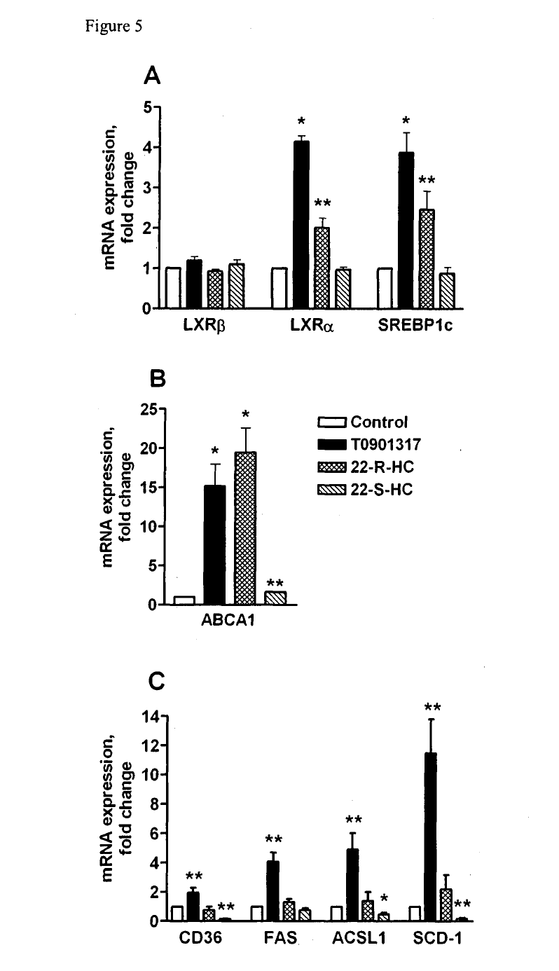

FIG. 5. Effects of 22-hydroxycholesterols on expression of LXR target genes in human myotubes. Human myoblasts were differentiated and treated with LXR ligands as described in FIG. 3. Total RNA were isolated from the cells, reversely transcribed and analyzed by Real-Time RT-PCR. Results are normalized to levels of 36B4 and present means±SEM (n=4-6; independent muscle cell donors). Relative expressions of (A) liver X receptor (LXR)β, LXRβ, and sterol regulatory element-binding protein (SREBP)1c, (B) ATP-binding cassette transporter (ABC)A1 (C) acyl-CoA synthetase long chain family member 1 (ACSL1), fatty acid transporter (CD36), fatty acid synthase (FAS), and stearoyl-CoA desaturase (SCD)-1. *p<0.05 vs control, **p<0.05 vs all other treatments.

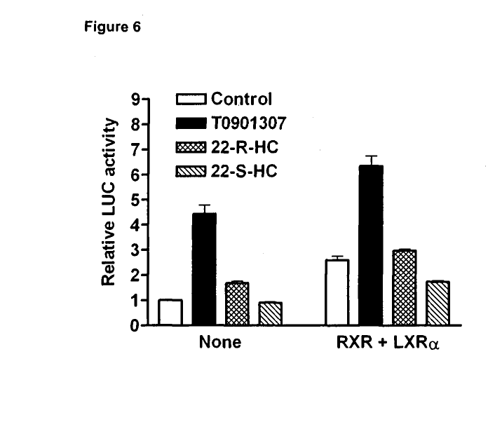

FIG. 6. Transfection with rat FAS promoter reporter shows LXR-dependent regulation for 22-hydroxycholesterols. COS-1 cells were transient transfected with rat FAS luciferase reporter and co-transfected with β-galactosidase (internal control), RXRα and LXRα expression vectors. Medium was supplied with vehicle (0.1% DMSO), 1 μM T0901317, 10 μM 22-R-hydroxycholesterol (22-R-HC) or 10 μM 22-S— hydroxycholesterol (22-S-HC) for 48 h. The results represent one of two experiments performed with triplicate cell culture dishes and are presented as means±SD.

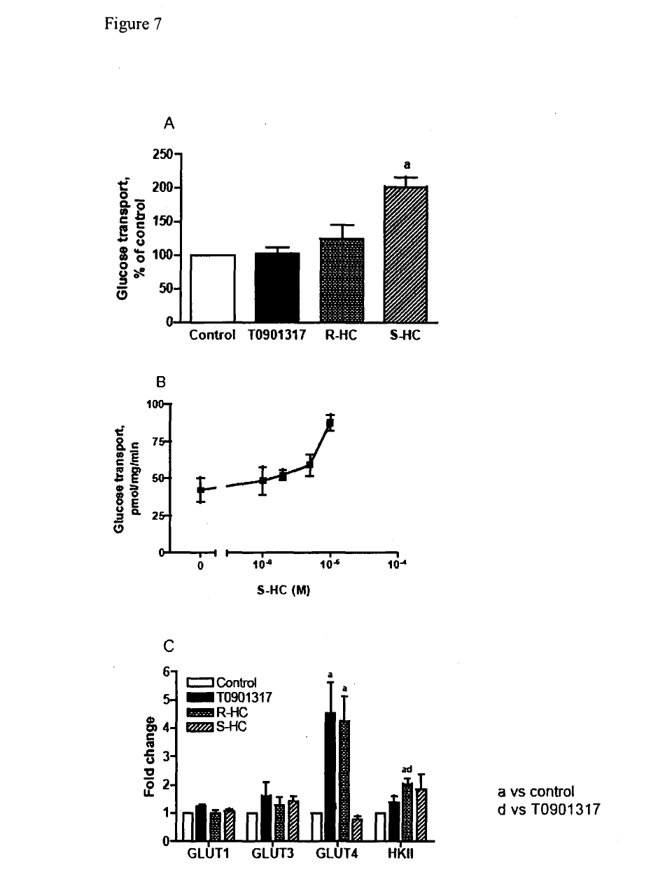

FIG. 7. Glucose transport is increased in human myotubes after chronic 22-S-HC treatment. Human myoblasts were allowed to differentiate for 2 days, and then exposed to 1 μmol/1 T0901317, 10 μmol/l 22-R-HC(R-HC) or 1, 2, 5 and 10 μmol/1 22-S-HC(S-HC) for 4 days. The cells were then incubated with [³H]2-deoxy-D-glucose (1.0 μCi/ml, 10 μmol/l) for 15 min. (A) Present means for glucose uptake±SEM (n=4). Basal level for control are 1.20±0.24 nmol/mg cell protein. (B) A representative dose-response curve for glucose uptake after 1, 2, 5 and 10 μmol/l 22-S-HC treatment (n=3). (C) Total RNA were reversely transcribed and analyzed by Real-Time RT-PCR. Results are normalized to levels of 36B4 and present means±SEM (n=4-6). Relative expressions of GLUT1, GLUT3, GLUT4, and hexokinase (HK)II, ^{a}p<0.05 vs control, ^{d}p<0.05 vs T0901317.

FIG. 8. This shows the structure of 22-S-HC.

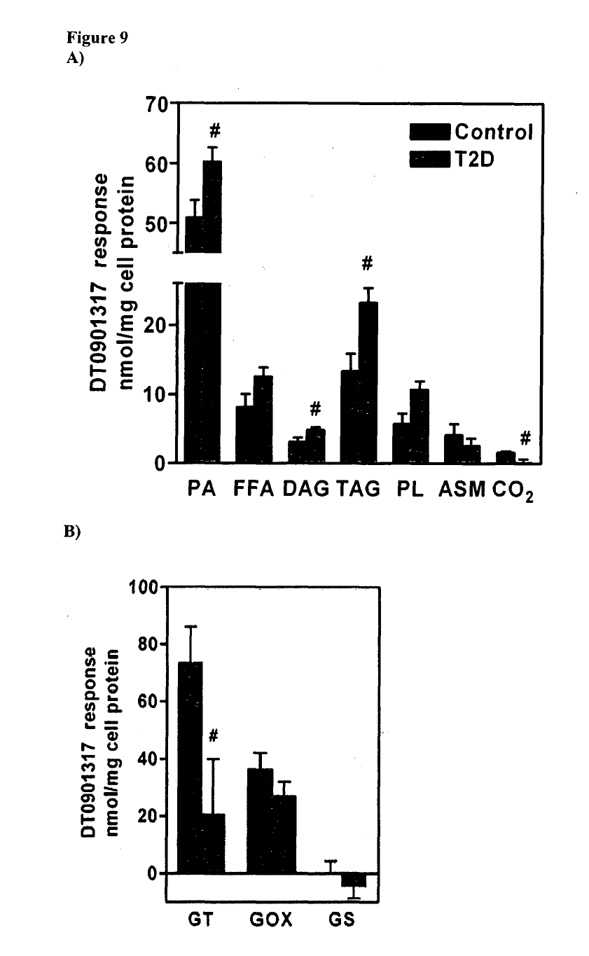

FIG. 9. Shows the effect on uptake and incorporation of palmitate into complex lipids and glucose uptake for T0901317 in myotubes from healthy and diabetic patients. T0901317 (1 μM) response of (A) palmitate uptake, distribution into cellular lipids and oxidation and (B) glucose uptake (GT), oxidation (GOX) and glycogen synthesis (GS) in myotubes from healthy donors and donors with type 2 diabetes #p≦0.05 vs control myotubes.

### EXAMPLE 1

### Preparation of a Prodrug of a 22-S-Hydroxycholesterol

A monoester mono-acid derivative of succinic acid and 22-S-hydroxycholesterol; i.e. H—C3O-cholesterol skeleton-22-S—O)—CO—(CH₂)₂—COONA, can be produced in one step by combining commercially available 22-S-hydroxycholesterol and succinic acid anhydride, adjusting the pH and purifying the compound.

### EXAMPLE 2

### Materials and Methods

Materials. Dulbecco's modified Eagle's medium (DMEM-Glutamax), foetal calf serum (FCS), Ultroser G, penicillin-streptomycin-amphotericin B, and trypsin-EDTA were obtained from Life Technology (Paisley, UK). [1-¹⁴C]acetic acid (54 mCi/mmol), [1-¹⁴C]palmitic acid (54 mCi/mmol) and 2-[³H(G)]deoxy-D-glucose (6 Ci/mmol) were purchased from American Radiolabeled Chemicals (St. Louis, Mo., USA). Insulin Actrapid was from Novo Nordisk (Bagsvaerd, Denmark). PA, bovine serum albumin (BSA) (essentially fatty acid-free), extracellular matrix (ECM) gel, 22-R-hydroxycholesterol and 22-S-hydroxycholesterol were purchased from Sigma Chem. Co. (St. Louis, Mo., USA). RNeasy Mini kit and RNase-free DNase were purchased from Qiagen Sciences (Oslo, Norway). The primers (36B4, ACSL1, ABCA1, CD36, FAS, GAPDH, GLUT4, LXRα, LXRβ, MyoD, myogenin, PPARα, PPARδ, PPARγ, SCD-1 and SREBP1c) were purchased from Invitrogen Corp. (invitrogen.comSted, Land?), while SYBR® Green and TaqMan reverse-transcription reagents kit were from Applied Biosystems (Warrington, UK). T0901317 was obtained from Cayman Chemical Company (Ann Arbor, Mich., USA). All other chemicals used were standard commercial high purity quality.

Human skeletal muscle cell cultures. A cell bank of satellite cells was established from muscle biopsy samples of the M. vastus lateralis of 6 healthy volunteers, age 25.7 years (±1.4), with BMI 21.6 (±1.0) and fasting glucose and insulin within normal range. The biopsies were obtained with informed consent and by approval of the National Committee for Research Ethics, Oslo, Norway. Muscle cell cultures free of fibroblasts were established by the method of Henry et al. (Diabetes, 44, 936-946, 1995), with minor modifications. Briefly, muscle tissue was dissected in Ham's F-10 media at 4° C., dissociated by three successive treatments with 0.05% trypsin/EDTA, and satellite cells were re-suspended in SkGM with 2% FCS and no added insulin. The cells were grown on culture wells coated with ECM gel (Apmis, 109, 726-734, 2001). At about 80% confluence, fusion of myoblasts into multinucleated myotubes was achieved by growth in DMEM with 2% FCS. All cells used were at passage 4 to 6. After 2 days in DMEM the cells were exposed to vehicle (0.1% DMSO), 1 μM T0901317, 10 μM 22-R-hydroxycholesterol (22-R-HC) or 10 μM 22-S-hydroxycholesterol (22-S-HC) for 4 days.

Palmitate uptake and lipid distribution. Myotubes were exposed to DMEM supplemented with 1.0 mM L-carnitine, [1-¹⁴C]palmitic acid (0.5 μCi/ml, 0.1 mM) for 4 h to study basal palmitate uptake and lipid distribution. Myotubes were placed on ice, washed three times with PBS (1 ml), harvested into a tube in 250 μM 0.05 M NaOH and homogenized. The radioactivity in the cell fraction (20 μl) was quantified by liquid scintillation (Packard Tri-Carb 1900 TR) (Gaster et al, Diabetes, 53, 542-548, 2004). The protein content of each sample was determined (Bradford, Anal. Biochem., 72, 248-254, 1976), and triacylglycerol (TAG) was extracted (Gaster et al, supra). Briefly, the homogenized cell fraction (220 μl) was extracted, lipids separated by thin-layer chromatography and the radioactivity was quantified by liquid scintillation.

Lipogenesis and lipid distribution. Myotubes were exposed to DMEM supplemented with [1-¹⁴C]acetic acid (2 μCi/ml, 0.1 mM) for 4 h to study lipogenesis and acetate incorporation into TAG and diacylglycerol (DAG). Myotubes were harvested and analyzed as described above (Palmitate uptake and lipid distribution).

RNA isolation and analysis of gene expression by RT-PCR. Myotubes were washed, trypsinized and pelleted before total RNA was isolated by RNeasy Mini kit (Qiagen Sciences, Oslo, Norway) or Agilent Total RNA Mini kit (Matrix, Oslo, Norway) according to the suppliers total RNA isolation protocol. RNA samples were incubated with RNase-free DNase (Qiagen Sciences) for minimum 15 min in an additional step during the RNA isolation procedure. Total RNA (1 μg/μl) was reversely transcribed with hexamere primers using a Perkin-Elmer Thermal Cycler 9600 (25° C. for 10 min, 37° C. for 1 h, 99° C. for 5 min) and a TaqMan reverse-transcription reagents kit (Applied Biosystems). Real time PCR was performed using an ABI PRISM® 7000 Detection System. DNA expression was determined by SYBR® Green (Applied Biosystems), and primers [36B4 (Acc#M17885), ACSL1 (Acc#NM_{—}001995), ABCA1 (Acc#AF165281), CD36 (Acc#L06850), FAS (Acc#U26644), GAPDH (Acc#J104038/M33197, GLUT4 (Acc#M20747), LXRα (Acc#U22662), LXR (Acc#U07132), Myogenin (Acc#X17651), MyoD (Acc#BC064493), PPARα (Acc#L02932), PPARδ (Acc#BC002715), PPARγ (Acc#L40904), SCD-1 (Acc#AB032261), SREBP1c (Acc#U00968)], GLUT1, (Acc#03195), GLUT3 (Acc#M20681), HKII (Acc#AF148513) were designed using Primer Express® (Applied Biosystems). Each target gene was quantified in triplicates and carried out in a 25 μl reaction volume according to the suppliers protocol. All assays were run for 40 cycles (95° C. for 12 s followed by 60° C. for 60 s). The transcription levels were normalized to the housekeeping control genes 36B4 and GAPDH.

Transfection and Luciferase Assay. Monkey kidney COS-1 cells (ATCC no. CRL 1650) were grown in DMEM supplemented with 10% FBS. For reporter gene assays, COS-1 cells were transiently transfected with luciferase reporter containing sequences from −1594 to +67 bp of the rat FAS promoter (5 μg), with a known LXR responsive element (LXRE) located at −669 to −655 (kindly provided by Peter Tontonoz, Howard Hughes Medical Institute, California, USA) and co-transfected with pCMX-RXRα, pCMX-LXRα, (1 μg each), and pSV-β-galactosidase (3 μg) expression vectors with calcium phosphate precipitation. Total DNA concentration was adjusted to 12 μg with corresponding empty expression vectors and pGL3-basic vector. After 3 h of transfection, medium containing appropriate reagents was added for 48 h. Cells were harvested in 100 μl lysis buffer, and luciferase activities were measured in a TD-20/20 Luminometer (Turner Designs, Sunnyvale, Calif.) using the dual luciferase assay kit (Promega). Relative luciferase activity was normalized against β-galactosidase activity.

Glucose Uptake. Skeletal biopsies were taken from M.vastus lateralis of healthy human donors and human myoblasts were allowed to differentiate for 4 days. On day 4, myotubes were exposed to T0901317 or 22-R-HC or 22-S-HC for another 4 days. Cultures were exposed to DMEM supplemented with 0.24 mmol/l BSA, 2-[³H]deoxy-D-glucose (0.2 μCi/well), and 25 pmol/l or 1 mol/l insulin for 15 min to study basal glucose uptake. Cells were solubilized by addition of 500 μl 0.1 mol/l NaOH. An aliquot (50 μl) was removed for protein determination (Bradford 1976) 300 μl was counted by liquid scintillation (Gaster and Beck-Nielsen 2004).

Statistical analysis. Data in text, tables and figures are given as mean (±SEM) and all experiments were run in triplicate. Comparison of different treatments were evaluated by paired Students T-test, and p<0.05 was considered significant. In FIG. 2, the curves are smoothed using the weighted average of five nearest neighbours (GraphPad Prism, ver. 3.02).

### Results

Expression of myogenic regulatory factors during differentiation of myoblasts into myotubes. Based on studies in mouse skeletal muscle, MyoD is required for the initiation of differentiation of myoblasts into myotubes. It is classified as a primary myogenic regulatory factor, while myogenin plays a major role during late differentiation and is classified as a secondary myogenic regulatory factor. Previous studies in mouse and rat skeletal muscle have shown that the expression of MyoD is induced from day 0-1 (start of differentiation=day 0), while the expression of myogenin peaked one day later. The present results show that expression levels of MyoD (FIG. 1A) and myogenin (FIG. 1B) peaked at day 0-2 to induce differentiation, followed by a rapid decline in the expression levels that remained low in mature myotubes. Further, to confirm that chronic treatment (4 days) with an LXR agonist does not alter or influence the differentiation process of myoblasts into myotubes, the expression of the muscle differentiation gene markers MyoD and myogenin were analyzed after treatment with 1 μM T0901317 from day 2 until day 6. The LXR agonist treatment did not seem to interfere with the expression levels of these genes (FIG. 1). Also, a normal differentiation of myoblasts into multinucleated myotubes with and without LXR agonist treatment was confirmed by light microscopy (data not shown).

Expression of known LXR target genes during differentiation of myoblasts into myotubes. Cultured human myoblasts were differentiated into myotubes at 70-80% confluence (day 0). Then the cells were harvested each day from day—2 until day 8 during the differentiation process. The LXRα and LXRβ genes were expressed early during differentiation and slightly increased in mature myotubes (FIG. 2A-B). The expression levels of SREBP1c and GLUT4 genes markedly peaked at day 2 and then declined in mature myotubes (FIG. 2C-D), while gene expression of FAS peaked at day—1 (FIG. 2E). Another subfamily of nuclear receptors, the peroxisome proliferator-activated receptors (PPARα, δ, γ) that are known key regulators of lipid and glucose homeostasis, were also studied. Like SREBP1c and GLUT4, PPARα gene expression peaked at day 2 and then declined (FIG. 2F). Gene expression of PPARδ (FIG. 2G) showed a pattern resembling the LXR genes, while the PPARγ gene that is a known marker of adipocyte differentiation was expressed highest between day—1 and 2, before its expression declined towards day 8 (FIG. 2H).

22-S-hydroxycholesterol decreases triacylglycerol synthesis. Human myoblasts were allowed to differentiate for 2 days and then exposed to 1 μM T0901317, 10 μM 22-R-HC or 10 μM 22-S-HC for another 4 days. As shown in FIG. 3, T0901317 increased TAG synthesis from labeled palmitate, whereas treatment with 22-S-HC showed a 50% reduction in incorporation of labeled palmitate into TAG when compared to control myotubes. Compared to T0901317 treatment, treatment with 22-S-HC significantly reduced synthesis of TAG (FIG. 3).

22-hydroxycholesterols influence lipid formation from acetate differently than the synthetic LXR agonist T0901317. The cells were incubated with labeled acetate to verify whether the LXR ligands could influence synthesis of free fatty acids (FFA), diacylglycerol (DAG) and TAG differently. The results show that FFA synthesis was 2-fold and 3-fold increased by T0901317 and 22-R-HC treatment, respectively, compared to control myotubes, while 22-S-HC only tended to increase FFA synthesis slightly (FIG. 4). Incorporation of labeled acetate into cellular TAG and DAG resulted in a different picture; T0901317 increased levels of DAG and increased TAG (FIG. 4). Further, 22-R-HC did not change the level of DAG compared to control myotubes and produced a slight increase in the level of TAG (which was not seen using cells from older donors-data not shown) whereas 22-S-HC showed a ˜50% reduction for DAG and a tendency towards reduced TAG (FIG. 4).

22-hydroxycholesterols regulate certain LXR target genes differently than the synthetic LXR agonist T0901317. Based on results obtained with the radiolabeled tracers the expression of certain genes important for lipid uptake and accumulation were examined after exposure to T0901317 and 22-HC. The expression of LXRα and SREBP1c (FIG. 5A) were 4-5-fold increased after T0901317 treatment and 2-3-fold increased after treatment with 22-R-HC. The expression level of the ATP-binding cassette transporter A1 (ABCA1) (FIG. 5B) increased 14-fold after T0901317 treatment, 17-fold after 22-R-HC treatment and very slightly (but insignificantly) after 22-S-HC treatment. The mRNA expression of fatty acid transporter CD36, FAS, ACSL1 and SCD-1 (FIG. 5C) were 2-fold, 4-fold, 5-fold and 10-fold increased by T0901317 treatment, respectively, but were unaffected after chronic exposure to 22-R-HC. The expression level of LXRbeta did not respond to any of the treatment regimes (FIG. 5A). None of the genes described in FIG. 5A-B were significantly affected by 22-S-HC treatment. However, this was not the case for CD36, ACSL1 and SCD-1 mRNA expression which were markedly down-regulated by ˜50-80% after chronic treatment with 22-S-HC (FIG. 5C). FAS mRNA expression was significantly reduced after 22-S-HC treatment when observed on a micro fluid card-data not shown. Compared to T0901317, both treatment with 22-R-HC and 22-S-HC significantly reduced mRNA expression of CD36, FAS, ACSL1 and SCD-1 (FIG. 5C).

Transfection with the Rat FAS Promoter

To further study whether oxysterols were able to activate LXR target genes through an LXRE located upstream in the FAS promoter, it was examined whether 22-R-HC and 22-S-HC were able to regulate the rat FAS gene. To study this a luciferase reporter construct that contains the rat FAS promoter (−1594 to +67 bp) with an LXRE was transiently transfected into COS-1 cells in combination with RXRα, and LXRα expression vectors and treated with LXR ligands (T0901317, 22-R-HC and 22-S-HC) (FIG. 6). A maximal 6.5-fold reporter activity was observed after addition of both receptor expression vectors and T0901317 (FIG. 6). Further, 22-R-HC was unable to regulate the FAS gene promoter while 22-S-HC seemed to reduce reporter activity compared to unstimulated control cells.

### Glucose Uptake

Experiments performed on myotubes from young, healthy subjects, showed quite surprisingly that basal glucose uptake increased 2-fold after exposure to 22-S-HC for 4 days. Glucose uptake tended to increase after 22-R-HC, but showed no effect after exposure to T0901317 (FIG. 7A). Basal glucose uptake increased in a dose—dependent manner after exposure to 0, 1.0, 2.0, 5.0 and 10 μmol/l 22-S-HC for 4 days (FIG. 7B). Analysis of mRNA levels for glucose transporter 1 (GLUT1) and 3 (GLUT3) showed no alterations for any treatments (GLUT1 tended to increase after T0901317, p=0.056), while GLUT4 increased 4-fold after exposure to T0901317 and 22-R-HC (FIG. 7C).

Myotubes from Type II Diabetic vs Healthy subjects

Myotubes from type II diabetic subjects showed an elevated uptake and incorporation of palmitate into complex lipids and reduced glucose uptake in response to activation of LXRs with T0901317, but an absence of palmitate oxidation to CO₂ compared to myotubes from healthy human donors (FIG. 9).

### Discussion

The present study confirmed that 22-R-HC is an active LXR ligand also in human myotubes, and showed that it can regulate expression of important LXR target genes controlling fatty acid metabolism and thereby modify lipid metabolism. Further, it was shown that 22-S-HC repressed the expression of certain genes and changed metabolic processes that resulted in reduced formation of complex lipids. Thus, 22-S-HC is not an inactive LXR ligand as previously suggested.

Monounsaturated fatty acids are important for living organisms because they are major constituents of complex lipids (phospholipids, triacylglycerols, cholesterol esters and alkyl-1,2-diacylglycerol). It has recently been shown by the inventors that chronic T0901317 treatment results in an increased uptake and incorporation of palmitate into complex lipids in myotubes (Kase et al, Diabetes, 54, 1108-1115, 2005). The role of 22-HC in lipid metabolism in human muscle cells has not previously been described. This study shows that in contrast to T0901317, 22-R-HC produced only a slight increase in TAG formation from acetate and did not increase formation of DAG from acetate, while 22-S-HC reduced both TAG synthesis from palmitate and formation of DAG from acetate when compared to control cells (FIGS. 3 and 4). Further, acetate incorporation into FFA increased after treatment with both T0901317 and 22-R-HC, whereas FFA levels only tended to increase after exposure to 22-S-HC (FIG. 4). We show in this study that important enzymes involved in lipid synthesis (ACSL1 and SCD-1) are reduced from control after 22-S-HC treatment. ACSL catalyzes the first step in intracellular lipid metabolism, the conversion of fatty acids to acyl-CoA thioesters. SCD-1 regulates the critical committed step in the biosynthesis of monounsaturated fatty acids from saturated fatty acids (e.g. palmitate) and is positively regulated by both cholesterol and LXR agonists. A recent report has shown that SCD−/− mice had reduced body adiposity, increased insulin sensitivity, were resistant to diet-induced obesity, while genes involved in lipid oxidation were up-regulated and lipid synthesis genes were down-regulated. Taken together, this strongly suggests that 22-S-HC reduces formation of DAG and TAG mainly by repressing the mRNA levels of SCD-1 and ACSL1.

Oxysterols, oxygenated derivatives of cholesterol, are intermediates or end products in cholesterol excretion pathways and are physiological mediators inducing a number of metabolic effects. LXRα may also be an important sensor of cholesterol metabolites. A cholesterol metabolite such as 22-R-HC has been reported to induce both the expression levels of ABCAI and SREBP1c in macrophages, fibroblasts and HepG2 cells. Further, Forman et al. (supra) have shown in fibroblasts (CV-1 cells) that 22-R-HC positively regulates LXRα, while 22-S-HC was reported to be inactive. We observed that 22-R-HC resulted in a similar response as T0901317 regulating LXRα target genes in lipid homeostasis, while the S-isomer was mainly inactive (FIG. 5A). However, the 22-hydroxycholesterols influenced the expression of certain genes (FAS, CD36, ACSL1 and SCD-1, FIG. 5C) involved in lipid uptake and handling differently than the synthetic LXR agonist; CD36, ACSL1 and SCD-1 were repressed by 22-S-HC, while 22-R-HC did not change their expressions levels. The 22-hydroxycholesterols both significantly reduced mRNA expression of CD36, FAS, ACSL1 and SCD-1 compared to T0901317 (FIG. 5C).

SREBPs are transcription factors central to the regulation of lipid homeostasis. They exist in three isoforms; SREBP1a, SREBP1c and SREBP2 and SREBP1c is probably the dominant isoform in skeletal muscle. Cholesterol metabolites have previously been described to inhibit the mature form of SREBPs. The genes (CD36, SCD, ACSL1) down-regulated by S-HC may also be regulated through SREBP1c and could therefore be down-regulated by an oxysterol-induced inhibition of SREBP1c maturation and not by interaction with LXR. However, recent data demonstrate that this sterol-sensitive process appears to be a major point of regulation of SREBP1a and SREBP2 isoforms, but not for SREBP1c. Such an oxysterol-induced inhibition does not explain the different regulation of lipid metabolism observed for 22-R-HC and 22-S-HC in this study. The transfection experiments confirmed that 22-S-HC can regulate the activity of the FAS gene through an LXRE located in the promoter differently than T0901317, supporting the assumption that 22-HC might regulate lipogenesis through direct interactions with LXR.

In summary, this study confirms that the endogenous 22-R-HC is a LXR agonist also in human myotubes and shows that it regulates lipid metabolism differently than T0901317. Furthermore, 22-S-HC is not an inactive LXR ligand in human myotubes. It seems to repress certain genes involved in lipogenesis and lipid handling that result in reduced synthesis of complex lipids.

### EXAMPLE 3

### Synthesis of 22(S)-Hydroxycholesterol Disuccinate

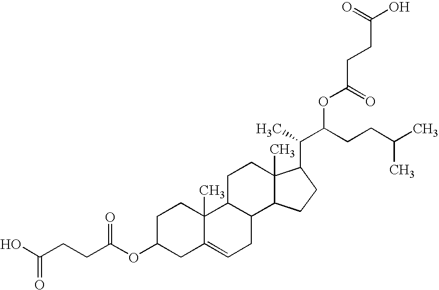

Succinic anhydride (200 mg, 2.0 mmol) was added to a solution of 22(S)-hydroxycholesterol (201 mg, 0.50 mmol) in pyridine (2 ml) and the mixture heated at 80° C. overnight, cooled to room temperature and evaporated in vacuo. The residue was added CH₂Cl₂ (5 ml) and the organic layer washed with water (3×2 ml), dried over anhydrous Na₂SO₄, filtered and the filtrate evaporated in vacuo to leave the title compound as a white solid.

¹H-NMR (200 MHz, DMSO-_{d6}):

δ12.17 (br s, 2H), 5.33 (d, 1H), 4.44 (d, 1H), 3.39-3.35 (m, 1H), 2.47-2.44 (m, 12H), 2.24 (d, 2H), 1.98-1.71 (m, 5H), 1.60-0.90 (m, 22H), 0.85-0.77 (m, 9H), 0.63 (s, 3)

MS (ES): 601 [M−H]⁺

### EXAMPLE 4

### Synthesis of 22(S)-Hydroxycholesterol Diglutarate

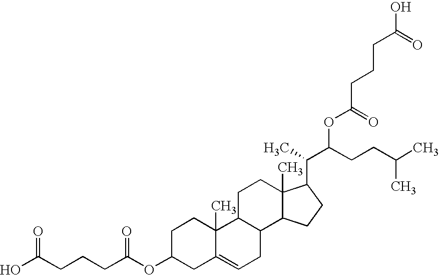

Glutaric anhydride (228 mg, 2.0 mmol) was added to a solution of 22(S)-hydroxycholesterol (201 mg, 0.50 mmol) in pyridine (2 ml) and the mixture heated at 80° C. overnight, cooled to room temperature and evaporated in vacuo. The residue was added CH₂Cl₂ (5 ml) and the organic layer washed with water (3×2 ml), dried over anhydrous Na₂SO₄, filtered and the filtrate evaporated in vacuo to leave the title compound as a white solid.

¹H-NMR (300 MHz, DMSO-_{d6}):

δ12.04 (br s, 2H), 5.33 (d, 1H), 4.46-4.44 (m, 1H), 3.38 (t, 1H), 3.35-3.20 (m, 1H), 2.28-2.20 (m, 8H), 1.85-1.66 (m, 10H), 1.44-0.96 (m, 22H), 0.84-0.78 (m, 9H), 0.63 (s, 3H)

MS (ES): 629 [M−H]⁺

### EXAMPLE 5

### Synthesis of 22(S)-Hydroxycholesterol Di-N-(Tert-Butoxycarbonyl)Glycinate

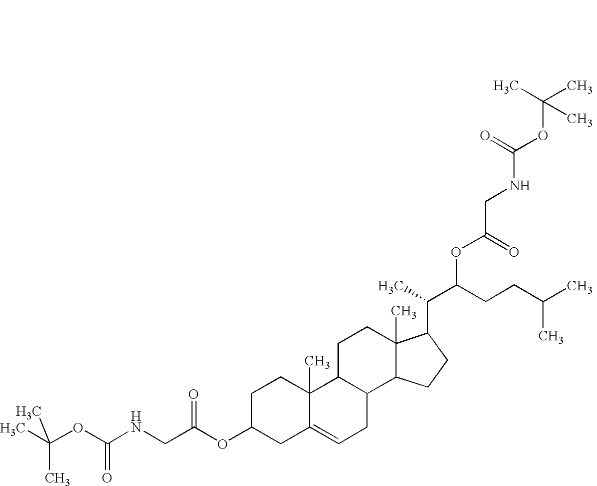

A mixture of N-(tert-butoxycarbonyl)glycine (192 mg, 1.10 mmol), N,N′-dicyclo-hexylcarbodiimide (227 mg, 1.10 mmol) and 22(S)-hydroxycholesterol (201 mg, 0.50 mmol) in CH₂Cl₂ (5 ml) at 0° C. was stirred to room temperature overnight, filtered and the filtrate washed with brine (2×1 ml) and water (1 ml). The organic layer was dried over anhydrous Na₂SO₄, filtered and evaporated in vacuo to leave the title compound as a white solid.

¹H-NMR (300 MHz, DMSO-_{d6}):

δ5.35 (d, 1H), 4.98 (t, 2H), 3.86 (t, 4H), 2.30 (d, 2H), 2.0-1.75 (m, 8H), 1.73-1.50 (m, 8H), 1.43 (s, 18H), 1.39-0.88 (m, 18H), 0.85 (s, 3H), 0.83

(s, 3H), 0.65 (s, 3H)

MS (ES): 716 [M−H]⁺

### EXAMPLE 6

### Synthesis of 22(S)-Hydroxycholesterol Diglycinate

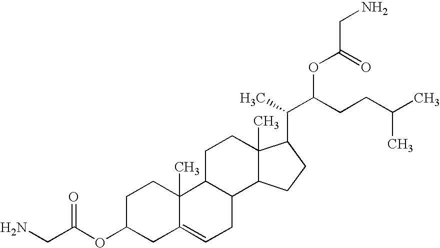

Trifluoracetic acid (5 ml) was added to a solution of 22(S)-hydroxycholesterol di-N-(tert-butoxycarbonyl)glycinate (235 mg, 0.33 mmol) in CH₂Cl₂ (5 ml) and stirred at room temperature overnight, evaporated in vacuo and the residue added CH₂Cl₂ (5 ml). The organic layer was washed with brine (3×1 ml) and water (1 ml), dried over anhydrous Na₂SO₄, filtered and evaporated in vacuo to leave the title compound as a yellow solid.

¹H-NMR (200 MHz, DMSO-_{d6}):

δ5.33 (d, 1H), 4.87 (t, 1H), 4.49-4.45 (m, 1H), 3.42-3.24 (m, 8H), 2.27

(d, 2H), 1.86-0.89 (m, 33H), 0.84-0.81 (m, 8H), 0.64 (s, 3H)

MS (ES): 517 [M+H]⁺

### EXAMPLE 7

### Stability of 22(S)-Hydroxycholesterol Diglutarate in Bovine Plasma

A stock solution of 22(S)-hydroxycholesterol diglutarate was prepared by adding 50 mg of the compound to a 50 ml volumetric flask, followed by 0.5 ml DMSO and deionised water to 50 ml, giving a final concentration of 1.0 mg/ml. The plasma solution was prepared by adding 0.38 ml of the 22(S)-hydroxycholesterol diglutarate stock solution to 1.62 ml of citrated bovine plasma, giving a final concentration of 300 μM. The citrated bovine plasma was incubated at 37° C. for 24 h. Proteins were discarded from the samples by centrifugal filtration before 50 μl volume of the filtrate was injected with an autosampler onto an analytical column (C18 reverse phase system) The mobile phase consisted of a gradient composed of water and acetonitrile (20→50% from 0 to 10 min, the mobile phase increased from 0.5 ml/min→1.0 ml/min in 10 minutes). Eluted compounds were detected at 210 nm. After incubation for 24 h it was not possible to detect 22(S)-hydroxycholesterol diglutarate in the bovine plasma solution.

### EXAMPLE 8

### Preparation of 22(S)-Hydroxycholesterol Diglycine Dimesylate Salt

Methanesulfonic acid (76 mg, 0.79 mmol) was added to a solution of 22(S)-hydroxycholesterol diglycinate (170 mg, 0.33 mmol) in CH₂CL₂ (2 ml). The solution was stirred at room temperature for ½ h, evaporated in vacuo to leave the title compound as a solid.

### EXAMPLE 9

### Preparation of 22(S)-Hydroxycholesterol Diglutarate Dimeglumine Salt

N-Methyl-D-glucamine (96 mg, 0.49 mmol) was added to a suspension of 22(S)-hydroxycholesterol diglutarate (130 mg, 0.20 mmol) in water (2 ml) and the reaction mixture heated at 60° C. for 2 h, cooled to room temperature and freeze dried to leave the title compound as a white crystalline solid.

### EXAMPLE 10

### Powder for Injection

22(S)-Hydroxycholesterol diglutarate dimegumine salt (from Example 7) (10 mg) is dissolved in water for injection (10 ml) and sterile filtered (0.22 micrometre) into a sterile injection vial (10 ml). The vial is freeze dried and sealed. Saline (10 ml) is added to the vial before use.

### EXAMPLE 11

### Tablets Comprising 3-Methoxy-22(S)-Hydroxycholesterol Phenylglyoxalate

3-methoxy-22(S)-hydroxycholesterol phenylglyoxalate (CAS NO. 896442-04-9) is prepared according to Tsuda et al. in J. Am. Chem. Soc (1959) 81, 5987.

| | | 100 000 |
|---|---|---|
| | One tablet | Tablets |
| 3-methoxy-22(S)-hydroxycholesterol | 20 mg | 2000 g |
| phenylglyoxalate | | |
| Microcrystalline cellulose (Avicel PH-101) | 600 mg | 60000 g |
| Magnesium stearate | 10 mg | 1000 g |
| Colloidal silica (Cab-O-Sil) | 2 mg | 200 g |

All ingredients are blended. Tablets are compressed using a Killian rotary tablet machine with 10 mm concave punch. 10 tablets weigh 6.32 g.

The produg is activated to release 22(S)-hydroxycholesterol by oxidative enzymes (probably CYP enzymes to remove the O-methyl group) and esterase (to release free 22(S)—OH group).

### EXAMPLE 12

### Tablets Comprising 22(S)-Hydroxycholesterol 22-Acetate

22(S)-hydroxycholesterol 22-acetate (CAS NO. 91509-28-3) is prepared according to JP 59027886 (Ihara Chemical Industry, Japan).

Tablets comprising 2 mg of the acetate are prepared as described in Example 11.

The prodrug is activated to release 22(S)-hydroxycholesterol by esterase.

### EXAMPLE 13

### Tablets Comprising 22(S)-Hydroxycholesterol Bis(Alpha-Methoxy-Alpha-(Trifluoromethyl)Benzeneacetate

22(S)-hydroxycholesterol bis(alpha-methoxy-alpha-(trifluoromethyl)benzeneacetate (CAS NO. 82033-37-2) is prepared according to Eguchi et al. in Heterocycles (1982), 17(Spec. Issue) 359.

Tablets comprising 20 mg of the diester are prepared as described in Example 11.

The prodrug is activated to release 22(S)-hydroxycholesterol by esterase.

### EXAMPLE 14

### Tablets Comprising 22(S)-Methoxycholesterol

22(S)-methoxycholesterol (CAS NO. 80320-69-0) is prepared according to Hirano et al. in Chem. Pharm. Bull. (1981), 29, 2254.

Tablets comprising 4 mg of the methoxy derivative are prepared as described in Example 11.

The prodrug is activated to release 22(S)-hydroxycholesterol by oxidative enzymes (probably CYP enzymes to remove the O-methyl group)

### EXAMPLE 15

### Tablets Comprising 22(S)-Hydroxycholesterol 3-(Tetrahydro-2H-Pyran-2-yl) Ether

22(S)-hydroxycholesterol 3-(tetrahydro-2H-pyran-2-yl)ether (CAS NO. 70116-50-6) ius prepared according to Ishiguro et al. in Chem. Pharm. Bull. (1978), 26, 3715.

Tablets comprising 5 mg of the ether derivative are prepared as described in Example 11.

The prodrug is activated to release 22(S)-hydroxycholesterol by hydrolysis or enzymatic cleavage of the acetal-ether.

### EXAMPLE 16

### Tablets Comprising 22(S)-Hydroxycholesterol Diacetate

22(S)-hydroxycholesterol diacetate (CAS NO. 17955-05-4) is prepared according to Burrows et al. in J. Org. Chem. (1969), 34, 103.

Tablets comprising 50 mg of the diacetate derivative are prepared as described in Example 11.

The prodrug is activated to release 22(S)-hydroxycholesterol by esterase.

### EXAMPLE 17

### Tablets Comprising 22(S)-Hydroxycholesterol Dibenzoate

22(S)-hydroxycholesterol dibenzoate (CAS NO. 17955-01-0) is prepared according to Burrows et al. in J. Org. Chem. (1969), 34, 103.

Tablets comprising 10 mg of the dibenzoate derivative are prepared according to Example 11.

The prodrug is activated to release 22(S)-hydroxycholesterol by esterase.

### EXAMPLE 18

### Tablets Comprising 22(S)-Hydroxycholesterol 3-Benzoate

22(S)-Hydroxycholesterol (17954-95-9) is prepared according to Burrows et al. in J. Org. Chem. (1969), 34, 103.

Tablets comprising 5 mg of the ester derivative is prepared according to Example 11.

### EXAMPLE 19

### Tablets Comprising 22(S)-Hydroxycholesterol Diacetate and Rosigitazon

Tablets comprising 22(S)-hydroxycholesterol diacetate (5 mg) and rosiglitazon (4 mg) (as the maleate salt) is prepared as described in Example 11.

1. A method of treating a subject to combat insulin resistance or a disorder associated therewith comprising administering 22-S-hydroxycholesterol, or a physiologically-acceptable pro-drug therefor to said subject.

2. The method as claimed in claim 1, wherein said insulin resistance or a disorder associated therewith is insulin resistance.

3. The method as claimed in claim 1, wherein said insulin resistance or a disorder associated therewith is type II diabetes.

4. The method as claimed in claim 1, wherein said insulin resistance or a disorder associated therewith is obesity.

5. The method as claimed in claim 1, wherein said insulin resistance or a disorder associated therewith is tissue lipid accumulation.

6.-8. (canceled)

9. The method as claimed in claim 1, wherein said prodrug is transformed in vivo to 22-S-hydroxycholesterol by an enzymatic transformation or a hydrolytic reaction.

10. The method as claimed in claim 1, wherein the prodrug is transformed in vivo to 22-S-hydroxycholesterol by esterases, amidases and/or oxidative enzymes.

11. The method as claimed in claim 1, wherein the prodrug comprises at least one functional group selected from the group consisting of esters including carbonate esters, carbamates, ethers, acetals and alkoxy derivatives.

12. The method as claimed in claim 1, wherein the prodrug is a compound of formula I or a physiologically acceptable salt thereof:
L)ₙ-O—CH₂—OW Ia
L)ₙ-O—W Ib
L)ₙ-O—CO(NH)ₐ—(O)_{b}—W Ic
wherein,
L-OH is 22-S-hydroxycholesterol,
n is a positive integer or a positive fraction,
a=0 or 1, b=0 or 1 and a+b=0 or 1, and
W is a linear, branched or cyclic, saturated or unsaturated organic group that comprises up to 25 carbon atoms and optionally incorporates heteroatoms (e.g. O, N and/or S).

13. The method as claimed in claim 1, wherein the prodrug is a compound of Formula II:
L)ₙ-O—CO(NH)ₚ—R—XY (Formula II)
wherein
L-OH=22-S-hydroxycholesterol;
n=positive integer or a positive fraction;
p=0 or 1
HO—CO R(X)ₘ Y=an acid or salt, amide or ester thereof; or
HO—CONHR(X)ₘY=an acid or salt, amide or ester thereof;
X=a solubilizing group;
m=zero or positive integer;
R is a linear, branched or cyclic, saturated or unsaturated organic group and may comprise up to 25 carbons, optionally incorporating heteroatoms;
Y=hydrogen(s) or a physiologically tolerable counterion(s) or a hydrophobic amino alcohol possessing a cation on the amino group at physiological pH.

14. The method as claimed in claim 1, wherein the prodrug is a compound of formula III or a physiologically acceptable salt thereof: 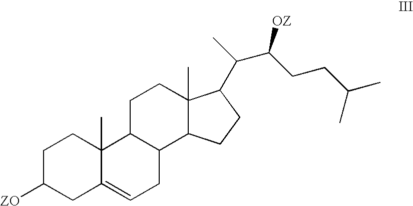
wherein,
each Z may be the same or different and is selected from H or CO(NH)ₐ—(O)_{b}—W wherein a=0 or 1, b=0 or 1 and a+b=0 or 1 and W is a linear, branched or cyclic, saturated or unsaturated organic group that comprises up to 25 carbon atoms and optionally incorporates heteroatoms (e.g. O, N and/or S)

15. The method of claim 12 wherein W is alkyl (e.g. C₁₋₆ alkyl) or is a C₁₋₆ alkylene chain substituted by an COOH or NH₂ group.

16. The method of claim 12, wherein n is ½ or 1 or 2.

17. The method of claim 13, wherein m is 1-6, especially 1.

18.-24. (canceled)

25. A product comprising 22-S-hydroxycholesterol or a physiologically acceptable prodrug therefor, and a second agent effective for combating insulin resistance or a disorder associated therewith as a combined preparation for simultaneous, separate or sequential use in combating insulin resistance or a disorder associated therewith.

26. The product of claim 25 wherein said second agent is selected from the group consisting of an inhibitor of cholesterol synthesis, a modulator of the peroxisome proliferators-activated receptor (PPAR), a sulfonylurea, an agent effective in the treatment of obesity, an agent affecting the angiotensin-renin system, an angiotensin II receptor antagonist, an anti-inflammatory agent, a cholinesterase inhibitor and an N-methyl D-aspartate (NDMA) receptor antagonist.

27. The method of claim 14 wherein W is alkyl (e.g. C₁₋₆ alkyl) or is a C₁₋₆ alkylene chain substituted by an COOH or NH₂ group.

28. The method of claim 13 wherein n is ½ or 1 or 2.

29. The method of claim 28, wherein m is 1-6, especially 1.


SEQUENCE LISTING (2 sequences)

SEQ ID NO: 1 - protein, 11 aa, Artificial Sequence
  Modified polyhistidine tag
MKKGHHHHHHG

SEQ ID NO: 2 - protein, 25 aa, Artificial Sequence
  Amino acids 675-699 of SRC1
CPSSHSSLTERHKILHRLLQEGSPS